(19) United States
(12) Patent Application Publication
(10) Pub. No.: US 2015/0159225 A1
(43) Pub. Date: Jun. 11, 2015

(54) UVEAL MELANOMA PROGNOSIS

(71) Applicant: THE CLEVELAND CLINIC FOUNDATION, Cleveland, OH (US)

(72) Inventors: Pierre L. Triozzi, Shaker Hts., OH (US); Arun D. Singh, Solon, OH (US)

(21) Appl. No.: 14/561,614

(22) Filed: Dec. 5, 2014

Related U.S. Application Data
(60) Provisional application No. 61/912,207, filed on Dec. 5, 2013.

(51) Int. Cl. C12Q 1/68

(52) U.S. Cl. CPC C12Q 1/6886; C12Q 2600/178

(57) ABSTRACT

Methods and kits for monitoring or providing a prognosis for a subject having uveal melanoma are described. The methods include obtaining a biological sample from the subject, determining the expression level of one or more uveal melanoma-associated miRs and/or miR biogenesis factors in the biological sample, and characterizing the subject as high risk if one or more uveal melanoma-associated miRs and/or miR biogenesis factors are differentially expressed as compared with a corresponding control.

### CROSS-REFERENCE TO RELATED APPLICATIONS

This application claims priority to U.S. Provisional Application Ser. No. 61/912,207, filed on Dec. 5, 2013, which is hereby incorporated by reference in its entirety.

### GOVERNMENT FUNDING

The present invention was made with government support by grant RO1CA136776 from the National Cancer Institute, National Institutes of Health. The U.S. Government has certain rights in this invention.

### BACKGROUND

Epigenetic events mediated by microRNA (miR), small, non-coding RNA, are implicated in cancer development and progression, and tumor-associated miRs are under investigation as diagnostic and prognostic biomarkers. Ferracin et al., Expert Rev Mol Diagn.; 10:297-308 (2010). MicroRNA genes are first transcribed by RNA polymerase and then cleaved within the nucleus into 60 to 70 nucleotide pre-miRs by the endonuclease Drosha (RNASEN), in conjunction with a RNA binding protein, DiGeorge syndrome critical region gene 8 (DGCR8), and RNA helicases, p69 (DDX5) and p72 (DDX17). Exportin 5 (XPO5) transports pre-miRs into the cytoplasm, where they are cleaved by the endonuclease Dicer (DICER1), in conjunction with TAR RNA binding protein 2 (TARBP2), into 21 to 22 nucleotide mature miRs. These are integrated into a silencing complex, which includes TARBP2, endonucleases such as Argonaute 1 (EIF2C1), Argonaute 2 (EIF2C1), and HIWI, and other proteins such as Gemin4 and Gemin5. The silencing complex binds to double-stranded RNA and regulates gene expression either by direct destruction or by repression of translation of the targeted mRNA.

Melanoma of the eye's uveal tract is a rare cancer that leads to metastatic death in up to half of patients despite successful local therapy. There is limited information regarding the diagnostic and prognostic utility of miR expression in uveal melanoma. Specific miRs have been implicated in uveal melanoma development. Compared to normal choroid, upregulation of miR-20a, 106a, 17, 21, and 34a and downregulation of miR-145 and miR-204 have been observed in uveal melanoma. Yang et al., Sci China Life Sci. 54:351-8 (2011). There is evidence that miR-37 and miR-34a can act as tumor suppressors in uveal melanoma. Chen et al., Invest Ophthalmol Vis Sci.; 52:1193-9 (2011); Yan et al., Invest Ophthalmol Vis Sci. 2009; 50:1559-65. Worley et al. found six miRs were upregulated in 12 tumors expressing the “class 2” gene expression profile associated with a high risk of metastasis and 68 miRs upregulated in 12 tumor expressing the “class 1” profile associated with a low risk. The most significant discriminators of class 2 were upregulation of let-7b and miR-199a. Worley et. al., Melanoma Res.; 18:184-90 (2008). There is also limited information regarding miR biogenesis factors in uveal melanoma, which have also been implicated in cancer development and progression, and may have prognostic significance. Wortham et al., Oncogene; 28:4053-64 (2009).

There is a need for prognostic blood biomarkers in uveal melanoma. Circulating miRs are very stable in blood due in part to their incorporation in to microparticles and exosomes. Blood miRs are under investigation as diagnostic and prognostic biomarkers in cancer and other diseases because of sensitive detection methods and their low complexity, when compared to proteins and cells.

### SUMMARY OF THE INVENTION

Uveal melanoma leads to metastatic death in up to half of patients despite successful local therapy. MicroRNAs (miRs) are under investigation as prognostic biomarkers. The association of tumor and plasma miRs with tumor monosomy-3, a chromosomal abnormality associated with the development of metastatic death, was evaluated.

Thirty-three uveal tumors obtained at enucleation with monosomy-3 and 22 without were subjected to genome-wide miR and mRNA expression profiling using Illumina arrays. Plasma miR profiles of 10 patients with and 10 without tumor monosomy-3 were determined using a quantitative nuclease protection assay array. Plasma levels of select differentially expressed miRs were quantified using quantitative real time polymerase chain reaction (qRT-PCR) in 33 patients with and 32 without tumor monosomy-3. Plasma levels were also compared to those of normal controls.

Six miRs were found to be overexpressed and 19 underexpressed in tumors manifesting monosomy-3. Tumors manifesting monosomy-3 were characterized by lower levels of DDX17 and TARB2 and by higher levels of XPO5 and HIWI, miR biogenesis factors. Levels of 11 miRs were elevated and four were reduced in the plasma of patients with tumor monosomy-3 compared to without. None of the miRs differentially expressed in the tumor array were differentially expressed in the plasma array. Only three of the 26 miRs identified as differentially expressed in the tumor arrays were detectable in plasma. Elevated plasma levels of miR-92b, identified in the tumor array, and 199-5p and miR-223, identified in the plasma arrays, in patients with tumor monosomy-3 was confirmed by qRT-PCR. Levels of these miRs were also higher in patients compared to normal controls.

These results support the analysis of tumor and blood miRs as biomakers of uveal melanoma metastatic risk. They also suggest that potentially useful blood miRs may be derived from host cells.

### BRIEF DESCRIPTION OF THE FIGURES

The present invention may be more readily understood by reference to the following figures, wherein:

FIG. 1 provides a graph showing miR biogenesis factor in enucleated tumors with (M), n=33, and without (D), n=22, tumor monosomy 3. The box represents with 25^{th} and 75^{th} percentiles, horizontal lines represent the median, and whiskers represent the minimum and maximum. Brackets with the P value above indicate statistical significance.

FIG. 2 provides a graph showing plasma miR quantification by qRT-PCR in patients with enucleated tumors with (M), n=10, and without (D) tumor monosomy 3, n=10. The horizontal lines represent the mean. Brackets with the P value above indicate statistical significance.

FIG. 3 provides a graph showing plasma miR quantification by qRT-PCR in patients with (M), n=33, without (D) monosomy 3, n=32, in which tumor chromosome 3 status was obtained on FNA biopsies. Also displayed are plasma levels of normal controls (N), n=26. The horizontal lines represent the mean. Brackets with the P value above indicate statistical significance.

### DETAILED DESCRIPTION OF THE INVENTION

The present invention relates methods of providing a prognosis, or monitoring the status of, a subject diagnosed as having uveal melanoma using microRNA profiling. The method allows use of a blood test, which avoids the need for conducting a biopsy of the eye. Identifying subjects having a high risk of developing metastasis can guide therapy for the subject.

### DEFINITIONS

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis).

As used herein, the term “prognosis” refers to a prediction of the probable course and outcome of a disease, or the likelihood of recovery from a disease. Prognosis is distinguished from diagnosis in that it is generally already known that the subject has the disease, although prognosis and diagnosis can be carried out simultaneously. In the case of a prognosis for uveal melanoma, the prognosis categorizes the relative severity of the uveal melanoma, and in particular the risk of metastasis, which can be used to guide selection of appropriate therapy for the uveal melanoma.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

The term therapy, as used herein, encompasses activity carried out to treat a disease. The specific activity carried out to conduct therapy can include use of surgery, radiotherapy, hormonal therapy, chemotherapy, or the use of one or more therapeutic agents (e.g., anticancer agents).

The terms “therapeutically effective” and “pharmacologically effective” are intended to qualify the amount of an agent which will achieve the goal of improvement in disease severity and the frequency of incidence over treatment of each agent by itself, while avoiding adverse side effects typically associated with alternative therapies. The effectiveness of treatment may be measured by evaluating a reduction in tumor load or decrease in tumor growth in a subject in response to the administration of anticancer agents. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

As used herein, the term “expression level,” particularly as applied to microRNA, refers to the absolute amount or relative amount of the microRNA in the sample. According to certain embodiments of the present disclosure, the term “expression level” means the normalized level of the microRNA. Expression levels may be normalized with respect to the expression level of one or more reference (housekeeping) microRNAs (e.g., an internal control microRNA), or the expression level may be normalized using global median normalization methods. Persons having ordinary skills in the art would recognize that numerous methods of normalization are known, and could be applied for use in the methods described herein. “Differential expression,” as used herein, refers to quantitative differences in the expression of microRNA in comparison to corresponding controls. The degree of a decrease or increase in miR expression can be any percentage value. For example, it can be 25% or more, 50% or more, or 75% or more as a percentage relative to a control, or corresponding reductions in expression.

“Expression profile” as used herein may mean a genomic expression profile, e.g., an expression profile of microRNAs. Profiles may be generated by any convenient means for determining a level of a nucleic acid sequence e.g. quantitative hybridization of microRNA, labeled microRNA, amplified microRNA, cRNA, etc., quantitative PCR, ELISA for quantitation, and the like, and allow the analysis of differential gene expression between two samples. A subject or patient tumor sample, e.g., cells or collections thereof, e.g., tissues, is assayed. Nucleic acid sequences of interest are nucleic acid sequences that are found to be predictive, including the nucleic acid sequences provided above, where the expression profile may include expression data for 5, 10, 20, 25, or more of, including all of the microRNA described herein. The term “expression profile” may also mean measuring the abundance of the nucleic acid sequences in the measured samples.

The term “control” or “reference value,” as used herein, refers to a value that statistically correlates to a particular outcome when compared to an assay result. In preferred embodiments the reference value is determined from statistical analysis of studies that compare microRNA expression with known clinical outcomes. The reference value may be a threshold score value or a cutoff score value. Typically, a reference value will be a threshold above which one outcome is more probable and below which an alternative outcome is more probable.

“Nucleic acid” or “oligonucleotide” or “polynucleotide”, as used herein, may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single-stranded or double-stranded, or may contain portions of both double-stranded and single-stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

The term “microRNAs” as used herein refers to a class of small RNAs typically between 15 and 30 nucleotides long. microRNAs can refer to a class of small RNAs that play a role in gene regulation. In a preferred embodiment, a microRNA refers to a human, small RNA of 20, 21, 22, 23, 24, 25, or 26 nucleotides long. MicroRNA can also be referred to as miR or miRNA.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” refers to +/−10% deviation from the basic value.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “a microRNA” includes reference to one or more microRNA molecules, and so forth.

### Methods of Providing a Prognosis or Monitoring a Subject Having Uveal Melanoma

One aspect of the invention provides a method of providing a prognosis for a subject having uveal melanoma. The method includes obtaining a biological sample from the subject, determining the expression level of one or more uveal melanoma-associated miRs and/or miR biogenesis factors in the biological sample, and characterizing the subject as at an increased risk if one or more uveal melanoma-associated miRs and/or miR biogenesis factors are differentially expressed as compared with a corresponding control.

A subject identified as being at increased risk is a subject who has a more severe form of cancer than a typical subject. A more severe form of cancer can include cancers at a more advanced stage, or cancer's having an increased risk of developing metastasis. In the case of uveal melanoma, metastasis typically occurs through local extension and/or blood borne dissemination. The most common site of metastasis for uveal melanoma is the liver; the liver is the first site of metastasis for 80%-90% of ocular melanoma patients. Other common sites of metastasis include the lung, bones and just beneath the skin (subcutaneous). A subject at increased risk is more likely to suffer greater symptoms, including death, if not provided with effective treatment, as compared with a subject who is not at increased risk. In some embodiments, the level of increased risk can be characterized as a percentage increase relative to the risk of a typical subject having uveal melanoma. For example, a subject at increased risk can be at a 10%, 20%, 30%, 40%, 50%, 60%, 70%, 80%, 90%, 100%, or greater than 100% level of increased risk as compared with a typical subject.

The prognosis is obtained by determining the expression level of one or more uveal melanoma-associated miRs and/or miR biogenesis factors. Uveal melanoma-associated miRs are miR that have been shown to be differentially expressed in a subject having uveal melanoma, and in particular differentially expressed in subjects having an increased risk of developing metastasis or a severe form of uveal melanoma. In some embodiments, individual miRs are shown to have increased or decreased expression in the subject, while in other embodiments a plurality of miRs or the expression profile of the miRs is changed or differentially expressed. For example, if a plurality of miR are evaluated, the expression of 2, 3, 4, 5, 10, 15, or 20 or more different miR can be evaluated. The nature of the differential expression can vary depending on the biological sample used to evaluate miR or miR biogenesis factor expression. The specific nucleotide sequence of particular miRs are known to those skilled in the art, and can readily be obtained through reference to the miRBase database of Cambridge University. Griffiths-Jones et al., Nucleic Acids Research, 34, D140-D144 (2006), the disclosure of which is incorporated herein by reference.

In some embodiments, the biological sample used to evaluate miR is a tumor sample; i.e., a sample obtained from the uveal melanoma itself. For example, in a tumor sample, the miRs miR-135a, miR-624, miR-449b, miR-142-5p, miR-92b, and miR-628-5p are over-expressed as compared with a control. Furthermore, the miRs miR-509-3-5p, miR-508-3p, miR 514, miR-506, miR-513a-5p, miR-507, miR-509-3p, miR-513b, miR-876-3p, miR-378, miR-935, miR-181a, miR-99a, miR-194, miR-592, miR-1296, miR-624, miR-140-5p, and miR-651 are under-expressed as compared with the corresponding control.

In other embodiments, the biological sample used to evaluate miR is blood, blood serum, or plasma. For example, in a blood, blood serum, or plasma sample, the miRs miR-191, miR-93, miR-221, miR-342-3p, miR-19b, miR-199a-5p, miR-25, miR-27a, miR-23a, miR-15b, and miR-223 are over-expressed as compared with the corresponding control, while the miRs miR-1227, miR-663, miR-654-5p, and miR-1238 are under-expressed as compared with the corresponding control.

In other embodiments, a miR biogenesis factor is detected when carrying out the method of providing a prognosis. MicroRNA biogenesis factors are miR processing factors that affect miR levels. Examples of miR biogenesis factors include TAR RNA binding protein 2, p′72, Exportin 5, and HIWI protein, or the corresponding genes expressing these factors; namely, TARB2, DDX17, XP05, and HIWI. In some embodiments, the biogenesis factor is TAR RNA binding protein 2 or p72 and the factor is upregulated, while in other embodiments the biogenesis factor is Exportin 5 or HIWI protein and the factor is dowregulated, as compared with a corresponding control.

Another aspect of the invention provides a method of monitoring the development of metastases in a subject having uveal melanoma. The method includes the steps of obtaining a blood, blood plasma, or blood serum sample from the subject, determining the expression level of one or more uveal melanoma-associated miRs in the sample, and characterizing the subject as having an increased risk of developing metastases if one or more uveal melanoma-associated miRs are differentially expressed as compared with a corresponding control. Monitoring is therefore similar to obtaining a prognosis. However, unlike a prognosis, monitoring is often carried out after treatment has been administered. Accordingly, in some embodiments, the subject being monitored has received anticancer treatment prior to the step of obtaining a sample. Furthermore, monitoring may involve repeating the steps of obtaining a sample and determining expression levels a plurality of times, to periodically check on the status of a subject having uveal melanoma. In some embodiments, the miR evaluated for monitoring are selected from the group consisting of miR-20a, miR-125b, miR-146a, miR-155, miR-223, miR-199a, and miR-181a.

### Uveal Melanoma

The present invention provides methods for prognosis, monitoring, and guiding treatment of uveal melanoma. Melanoma is a type of skin cancer which forms from melanocytes, and is typically caused by ultraviolet radiation. Uveal melanoma, which is also known as ocular melanoma, is a melanoma of the eye involving the iris, ciliary body, or choroid, which are collectively referred to as the uvea. In the case of large tumors, the melanoma may occupy portions of two or all of these regions. Tumors arise from the melanocytes that reside within the uvea giving color to the eye. Uveal melanoma does not include benign melanocytic tumors, such as iris freckles and moles (nevi), which are common and pose no health risks, unless they show signs of malignancy, in which case they are classified as iris melanomas.

As used herein, the terms “tumor” or “cancer” refer to a condition characterized by anomalous rapid proliferation of abnormal cells of a subject. The abnormal cells often are referred to as “neoplastic cells,” which are transformed cells that can form a solid tumor. The term “tumor” refers to an abnormal mass or population of cells (e.g., two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize, which refers to the spread of the cancer to other tissue sites.

Several clinical and pathological prognostic factors have been identified that are associated with higher risk of metastasis of uveal melanomas. In some embodiments, the method of providing a prognosis for a subject with uveal melanoma may include evaluation of one or more of these additional factors. These include large tumor size, ciliary body involvement, presence of orange pigment overlying the tumor, and older patient age. Augsburger J J, Gamel J W, Cancer 66 (7): 1596-1600 (1990). Likewise several histological and cytological factors are associated with higher risk of metastasis including presence and extent of cells with epithelioid morphology, presence of looping extracellular matrix patterns, increased infiltration of immune cells, as well as staining with several immunohistochemical markers. Pardo et al., Expert Rev Proteomics 4 (2): 273-286 (2007).

An important genetic alteration associated with poor prognosis in uveal melanoma is inactivation of BAP1, which most often occurs through mutation of one allele and subsequent loss of an entire copy of Chromosome 3 (Monosomy 3) to unmask the mutant copy. Harbour et al., Science 330 (6009): 1410-1413 (2010). Because of this function in inactivation of BAP1, monosomy 3 correlates strongly with metastatic spread. Prescher et al., Lancet 347 (9010): 1222-1225 (1996). Accordingly, in some embodiments, an additional prognostic factor is the presence of monosomy 3. Where BAP1 mutation status is not available, gains on chromosomes 6 and 8 can be used to refine the predictive value of the Monosomy 3 screen, with gain of 6p indicating a better prognosis and gain of 8 q indicating a worse prognosis in disomy 3 tumors. Damato et al., Invest Ophthalmol Vis Sci 50 (7): 3048-55 (2009). Monosomy 3, along with other chromosomal gains, losses, amplifications, can be detected in fresh or paraffin embedded samples by virtual karyotyping.

### Biological Samples

“Biological sample” as used herein means a sample of biological tissue or fluid that comprises nucleic acids. Such samples include, but are not limited to, tissue or fluid isolated from subjects. Biological samples may also include sections of tissues such as biopsy and autopsy samples, FFPE samples, frozen sections taken for histological purposes, blood, plasma, serum, sputum, stool, tears, mucus, hair, and skin. Biological samples also include explants and primary and/or transformed cell cultures derived from animal or patient tissues.

Biological samples may also be blood, a blood fraction, urine, effusions, ascitic fluid, saliva, cerebrospinal fluid, cervical secretions, vaginal secretions, endometrial secretions, gastrointestinal secretions, bronchial secretions, sputum, cell line, tissue sample, cellular content of fine needle aspiration (FNA) or secretions from the breast. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used. Biological samples may also be stored in RNAlater™ for analysis at a later date.

The microRNA identified can vary depending on the type of biological sample obtained. Accordingly, because the inventors have carried out a number of experiments using tumor tissue, blood, and plasma, in which the specific microRNA in these samples were characterized, in some embodiments the biological sample is blood, blood plasma, or blood serum, while in other embodiments the biological sample is a tumor sample.

The methods involve providing or obtaining a biological sample from the subject, which can be obtained by any known means including needle stick, needle biopsy, swab, and the like. In an exemplary method, the biological sample is a blood sample, which may be obtained for example by venipuncture.

A biological sample may be fresh or stored. Biological samples may be or have been stored or banked under suitable tissue storage conditions. The biological sample may be a tissue sample expressly obtained for the assays of this invention or a tissue sample obtained for another purpose which can be subsampled for the assays of this invention. Preferably, biological samples are either chilled or frozen shortly after collection if they are being stored to prevent deterioration of the sample.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC) or HPLC, or precipitation of proteins with dextran sulfate or other methods. Any of a number of standard aqueous buffer solutions at physiological pH, such as phosphate, Tris, or the like, can be used.

### Subjects

The terms “individual,” “subject,” and “patient” are used interchangeably herein irrespective of whether the subject has or is currently undergoing any form of treatment. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Prognosis or monitoring of humans is of particular interest.

Methods for Detecting microRNA or miR Biogenesis Factors

The expression level of the microRNA may be determined by any method known in the art, examples of which include but are not limited to amplification-based methods such as polymerase chain reaction (PCR), quantitative RT-PCR (qPCR), real-time quantitative PCR (RT-qPCR), semi-quantitative RT-PCR, ligase chain reaction (LCR), quantitative nuclease protection assay (qNPA), in situ hybridization, and strand displacement amplification (SDA).

In some embodiments, the microRNA is detected by hybridization with another nucleic acid sequence. The type of the nucleotide of the nucleic acid sequence is not particularly limited provided that it can specifically hybridize to the microRNA of the present invention. The length of the part of the polynucleotide is not particularly limited provided that it specifically hybridizes to the predetermined microRNA according to the present invention; however, it is preferably 10 to 100 mers, more preferably 10 to 40 mers in view of securing the stability of hybridization. The polynucleotide or a part thereof can be obtained by chemical synthesis or the like using a method well known in the art.

In some embodiments, the microRNA is detected by quantitative polymerase chain reaction. The quantitative PCR method is not particularly limited provided that it is a method using a primer set capable of amplifying the sequence of the microRNA and can measure the expression level of the present microRNA; conventional quantitative PCR methods such as an agarose electrophoresis method, an SYBR green method, and a fluorescent probe method may be used. However, the fluorescent probe method is most preferable in terms of the accuracy and reliability of quantitative determination.

The primer set for the quantitative PCR method means a combination of primers (polynucleotides) capable of amplifying the sequence of the microRNA. The primers are not particularly limited provided that they can amplify the sequence of the microRNA; examples thereof can include a primer set consisting of a primer consisting of the sequence of a 5′ portion of the sequence of a microRNA of the present invention (forward primer) and a primer consisting of a sequence complementary to the sequence of a 3′ portion of the microRNA (reverse primer). Here, the 5′ means 5′ to the sequence corresponding to the reverse primer when both primers were positionally compared in the sequence of a mature microRNA; the 3′ means 3′ to the sequence corresponding to the forward primer when both primers were positionally compared in the sequence of a microRNA.

Preferred examples of the 5′ sequence of a microRNA can include a sequence 5′ to the central nucleic acid of the microRNA sequence; preferred examples of the 3′ sequence of the microRNA can include a sequence 3′ to the central nucleic acid of the microRNA sequence. The length of each primer is not particularly limited provided that it enables the amplification of the microRNA; however, each primer is preferably a 7-to-10-mer polynucleotide. The type of the nucleotide of a polynucleotide as the primer is preferably DNA because of its high stability.

In some embodiments, the presence or amount of microRNA is determined using the quantitative nuclease protection assay. In this method, the extracted RNA is first mixed with antisense RNA or DNA probes that are complementary to the sequence or sequences of interest and the complementary strands are hybridized to form double-stranded RNA (or a DNA-RNA hybrid). The mixture is then exposed to ribonucleases that specifically cleave only single-stranded RNA but have no activity against double-stranded RNA. When the reaction runs to completion, susceptible RNA regions are degraded to very short oligomers or to individual nucleotides; the surviving RNA fragments are those that were complementary to the added antisense strand and thus contained the sequence of interest.

A person skilled in the art will appreciate that a number of detection agents can be used to determine the expression of the microRNA. For example, to detect microRNA, probes, primers, complementary polynucleotide sequences or polynucleotide sequences that hybridize to the microRNA can be used. In some embodiments, reverse complementary poylynucleotides serve as probes for microRNA. In alternate embodiments, a complementary polynucleotide sequence that hybridizes to the target polynucleotide sequence can be used to detect expression of the microRNA.

In some embodiments, a fluorescent probe is used. The fluorescent probe is not particularly limited provided that it comprises a polynucleotide consisting of a nucleic acid sequence complementary to the sequence of the present microRNA or a part thereof; preferred examples thereof can include a fluorescent probe capable of being used for the TaqMan™ probe method or the cycling probe method; the fluorescent probe capable of being used for the TaqMan™ probe method can be particularly preferably exemplified. Examples of the fluorescent probe capable of being used for the TaqMan™ probe method or the cycling probe method can include a fluorescent probe in which a fluorochrome is labeled 5′ thereof and a quencher is labeled on 3′ thereof. The fluorochrome, quencher, donor dye, acceptor dye used or the like used with a fluorescent probe are commercially available.

In some embodiments, the presence or amount of microRNA is determined using an array or microarray. The microarray method is not particularly limited provided that it can measure the level of the microRNA whose expression changes in response to the presence of uveal melanoma; examples thereof can include a method which involves labeling the RNA extracted from a tissue with a label (preferably a fluorescent label), contacting the RNA with a microarray to which a probe consisting of a polynucleotide (preferably DNA) consisting of a nucleic acid sequence complementary to the microRNA to be identified or a part thereof is fixed for hybridization, washing the microarray, and measuring the expression level of the remaining microRNAs on the microarray. The array to which the polynucleotide or a part thereof is fixed is not particularly limited; however, preferred examples thereof can include a glass substrate and a silicon substrate, and the glass substrate can be preferably exemplified. A method for fixing the polynucleotide or a part thereof to the array is not particularly limited; a well-known method may be used.

In other embodiments, the microarray is a biochip, sometimes referred to as an MMchip in the context of biochips designed for detecting microRNA. The biochip may comprise a solid substrate comprising an attached probe or plurality of probes described herein. The probes may be capable of hybridizing to a target sequence under stringent hybridization conditions. The probes may be attached at spatially defined locations on the substrate. More than one probe per target sequence may be used, with either overlapping probes or probes to different sections of a particular target sequence. The probes may be capable of hybridizing to target sequences associated with a single disorder appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrate materials include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as flexible foam, including closed cell foams made of particular plastics.

The substrate of the biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker. The probes may be attached to the solid support by either the 5′ terminus, 3′ terminus, or via an internal nucleotide. The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

In some embodiments, the presence or amount of one or more miR biogenesis factors is determined. The presence or levels of MicroRNA biogenesis factors can be determined by determining the presence or levels of the miR biogenesis factors themselves, or by determining level of RNA expressing the miR biogenesis factors. The presence and/or amount of the miR biogenesis factor(s) in a biological sample can be determined using polyclonal or monoclonal antibodies that are immunoreactive with the expressed protein. Use of antibodies comprises contacting a sample taken from the individual with one or more of the antibodies; and assaying for the formation of a complex between the antibody and a protein or peptide in the sample. For ease of detection, the antibody can be attached to a substrate such as a column, plastic dish, matrix, or membrane, preferably nitrocellulose. The sample may be untreated, subjected to precipitation, fractionation, separation, or purification before combining with the antibody. Interactions between antibodies in the sample and the miR biogenesis factor(s) are detected by radiometric, colorimetric, or fluorometric means, size-separation, or precipitation. Preferably, detection of the antibody-protein or peptide complex is by addition of a secondary antibody that is coupled to a detectable tag, such as for example, an enzyme, fluorophore, or chromophore. Formation of the complex is indicative of the presence of the miR biogenesis factor in the sample.

Antibodies immunospecific for miR biogenesis factors may be made and labeled using standard procedures and then employed in immunoassays to detect the miR biogeneic factors in a sample. Suitable immunoassays include, by way of example, immunoprecipitation, particle immunoassay, immunonephelometry, radioimmunoassay (RIA), enzyme immunoassay (EIA) including enzyme-linked immunosorbent assay (ELISA), sandwich, direct, indirect, or competitive ELISA assays, enzyme-linked immunospot assays (ELISPOT), fluorescent immunoassay (FIA), chemiluminescent immunoassay, flow cytometry assays, immunohistochemistry, Western blot, and protein-chip assays using for example antibodies, antibody fragments, receptors, ligands, or other agents binding the target analyte. Polyclonal or monoclonal antibodies raised against miR biogenesis factors are produced according to established procedures.

In some embodiments, the miR biogenesis factor(s) are detected using a method other than an immunoassay. For example, the miR biogenesis factor(s) can be detected using matrix-assisted laser desorption-ionization time-of-flight mass spectrometry (MALDI-TOF) or protein purification and analysis.

In other embodiments, the presence or amount of miR biogenesis factors is identified by determining the level of RNA expressing the miR biogenesis factors. Methods of determining the level of RNA expression include PCR, and other methods described herein for detecting nucleotides.

The miR and/or miR biogenesis factor proteins or nucleotides can be detected or measured by an analytic device such as a kit or a conventional laboratory apparatus, which can be either portable or stationary. In some embodiments, the levels of miR and/or miR biogenesis factor may be compared to the level of corresponding internal standards in the sample or samples when carrying out the analysis to quantify the amount of the miR or miR biogenesis factor being detected.

Once the presence and/or levels of the miR and/or miR biogenesis factors have been determined, they can be displayed in a variety of ways. For example, the levels can be displayed graphically on a display as numeric values or proportional bars (i.e., a bar graph) or any other display method known to those skilled in the art. The graphic display can provide a visual representation of the amount of the miR or miR biogenesis factor in the biological sample being evaluated.

### Therapeutic Methods

In some embodiments, the method of monitoring or prognosis can be used to guide treatment of the uveal melanoma. For example, the method can further comprise the step of providing suitable treatment if the subject is identified as being at increased risk. Suitable treatment can include use of a variety of different methods of treating cancer, such as surgery, radiation therapy, and administration of anticancer agents.

In some embodiments, treatment can involve removal of the affected eye (enucleation). However, unucleation is not preferred, and advances in radiation therapies have significantly decreased the number of patients treated by enucleation in developed countries. The most common radiation treatment is plaque brachytherapy, in which a small disc-shaped shield (plaque) encasing radioactive seeds (most often Iodine-125, though Ruthenium-106 and Palladium-103 are also used) is attached to the outside surface of the eye, overlying the tumor. The plaque is left in place for a few days and then removed. The risk of metastasis after plaque radiotherapy is the same as that of enucleation, suggesting that micrometastatic spread occurs prior to treatment of the primary tumor. Other modalities of treatment include transpupillary thermotherapy, external beam proton therapy, resection of the tumor, Gamma Knife stereotactic radiosurgery or a combination of different modalities. Different surgical resection techniques can include trans-scleral partial choroidectomy, and transretinal endoresection.

### Kits

The present disclosure also provides kits for providing a prognosis for a subject having uveal melanoma. The kits include one or more probes capable of determining the expression level of one or more uveal-melanoma-associated miRs and/or miR biogenesis factors in a biological sample, and a package for holding the primers or probes. A kit generally includes a package with one or more containers holding the reagents, as one or more separate compositions or, optionally, as an admixture where the compatibility of the reagents will allow. The kits may further include enzymes (e.g., polymerases), buffers, labeling agents, nucleotides, controls, and any other materials necessary for carrying out the extraction and/or detection of microRNA or miR biogenesis factors. Kits can also include a tool for obtaining a sample from a subject, such as a syringe or a punch tool to obtain a punch-biopsy or needle biopsy.

In some embodiments, the kit comprises one or more probes capable of determining the expression level of a miR selected from the group consisting of miR-135a, miR-624, miR-449b, miR-142-5p, miR-92b, and miR-628-5p, miR-509-3-5p, miR-508-3p, miR 514, miR-506, miR-513a-5p, miR-507, miR-509-3p, miR-513b, miR-876-3p, miR-378, miR-935, miR-181a, miR-99a, miR-194, miR-592, miR-1296, miR-624, miR-140-5p, and miR-651.

In other embodiments, the kit includes one or more probes capable of determining the expression level of a miR selected from the group consisting of miR-191, miR-93, miR-221, miR-342-3p, miR-19b, miR-199a-5p, miR-25, miR-27a, miR-23a, miR-15b, and miR-223, miR-1227, miR-663, miR-654-5p, and miR-1238.

In a further embodiment, the kit includes one or more probes capable of determining the expression level of a polynucleotide expressing miR biogenic factor selected from the group consisting of is TARB2, DDX17, XPO5 and HIWI.

“Probe”, as used herein, may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence, depending upon the stringency of the hybridization conditions. There may be any number of base pair mismatches which will interfere with hybridization between the target sequence and the single-stranded nucleic acids described herein. However, if the number of mutations is so great that no hybridization can occur under even the least stringent of hybridization conditions, the sequence is not a complementary target sequence. A probe may be single-stranded or partially single- and partially double-stranded. The strandedness of the probe is dictated by the structure, composition, and properties of the target sequence. Probes may be directly labeled or indirectly labeled such as with biotin to which a streptavidin complex may later bind.

The term “complementary” as used herein refers to nucleotide sequences that complement the polynucleotides' reverse sequence. Complementarity is the base principle of DNA replication and transcription as it is a property shared between two DNA or RNA sequences, such that when they are aligned antiparallel to each other, the nucleotide bases at each position in the sequences will be complementary. Complementarity is achieved by distinct interactions between nucleobases: adenine, thymine (uracil in RNA), guanine and cytosine. Adenine (A) and guanine (G) are purines, while thymine (T), cytosine (C) and uracil (U) are pyrimidines. Purines are larger than pyrimidines. Both types of molecules complement each other and can only base pair with the opposing type of nucleobase. In nucleic acid, nucleobases are held together by hydrogen bonding, which only works efficiently between adenine and thymine and between guanine and cytosine. The base complement A=T shares two hydrogen bonds, while the base pair G≡C has three hydrogen bonds.

The degree of complementarity between two nucleic acid strands may vary, from complete complementarity (each nucleotide is across from its opposite) to no complementary (each nucleotide is not across from its opposite) and determines the stability of the sequences to be together. Lesser degrees of complementarity are referred to herein by percentages of sequence identity as compared with a sequence having 100% complementarity. Embodiments of the invention include sequences having at least about 70% to at least about 100% sequence identify to a complementary sequence. For example, probes can have sequences having at least 70%, 75%, 80%, 85%, 90%, 95%, or 100% sequence identify with a complementary probe. In another example, the sequence identity can be at least about 80% to at least about 95% that of a complementary sequence. In a preferred embodiment, the probe can have at least about 87%, 88%, 89%, 90%, 91%, or 92% sequence identity to a complementary probe. In another preferred embodiment, the probe can have at least 90% sequence identity to a complementary probe.

In another aspect, kits for guiding or monitoring treatment are provided that include an array and/or microarray and oligonucleotide probes for identifying the presence or amount of a differentially expressed microRNA or miR biogenesis factor using PCR, qNPA, or another sequencing technology known to those skilled in the art.

The kit can also include instructions for using the kit to carry out a method of providing a prognosis or monitoring a subject having uveal melanoma. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

An example has been included to more clearly describe a particular embodiment of the invention and its associated cost and operational advantages. However, there are a wide variety of other embodiments within the scope of the present invention, which should not be limited to the particular example provided herein.

### EXAMPLE

### Example 1

### Tumor and Plasma microRNA Profiling of Patients with Uveal Melanoma with and without Tumor Monosomy 3

The inventors examined the miR expression profiles and miR biogenesis factors in tumors expressing the high risk monosomy 3. The miR expression profiles of the plasma of patients with uveal melanoma with and without tumor monosomy 3 were also examined. Plasma miR levels have not been previously reported in uveal melanoma.

### Materials and Methods

### Patients

Tumors that had been cryopreserved from patients with uveal melanoma treated by enucleation at the Cleveland Clinic Cole Eye Institute between 2004 and 2010 were analyzed. Starting in 2009 blood was also collected from patients treated with enucleation and from patients undergoing fine needle aspiration (FNA) biopsy at the time of plaque radiotherapy. Computed tomography scans of the chest, abdomen, and pelvis were initially performed to rule out metastatic disease. Chromosome 3 status in the enucleation specimen was assessed by single nucleotide polymorphism array and in the FNA biopsies by fluorescent in situ hybridization (FISH) as previously described. Singh et al., Invest Ophthalmol Vis Sci.; 53:3331-9 (2012). Standard clinical and histologic characteristics were also assessed. All patients underwent scheduled surveillance for the development of metastases every 6 months with clinical evaluation, liver imaging, and liver function testing. Studies were approved by the Cleveland Clinic Institutional Review Board, and all patients provided written informed consent. Plasma was also obtained from healthy donors without ocular disease, also on an approved study.

### Tumor miR Array

Total RNA was extracted from 25 mg of cryopreserved tumor tissue using Trizol Reagent (Invitrogen, Carlsbad, Calif.) according to the manufacturer's instructions, and further purified using the RNeasy Mini Kit (Qiagen, Valencia, Calif.). RNA quality was assessed using the Agilent 2100 Bioanalyzer and concentration was measured using Nanodrop 1000 (Thermo Fisher Scientific, Waltham, Mass.). Total RNA (400 ng) was hybridized to the Illumina MicroRNA Profiling BeadChip, containing 858 mature human miR probes and 287 hypothetical small RNA probes according to the standard protocol. To identify miRs that were differentially expressed between tumor subgroups, supervised analysis was performed using significance analysis of microarrays, available at the Stanford University website. Expression values for each array were median-centered and 1000 permutations were performed using the t-test statistic. A false discovery rate of Q=0.001 was set as the threshold for identification of differentially expressed miRs.

### Plasma miR Array

Pooled plasma samples were forwarded to HTG Molecular Diagnostics, Inc. (Tucson, Ariz.) for miR profiling using quantitative nuclease protection assay (qNPA) array. Each sample was tested in duplicate. Data were normalized to the total signal for each microarray. Results are reported as average signal intensities. miRs in the setting of monosomy-3 were considered to be detectable in plasma if average signal intensity was >526; disomy-3, if >531.

### Plasma miR Quantification

Total RNA was isolated from plasma using the miRNeasy Mini Kit (Qiagen, Valencia, Calif.) according to the manufacturer's instructions. With the exceptions of miR-142-5p and miR-92b, reverse transcription reactions were performed using a TaqMan MicroRNA Reverse Transcription Kit (Applied Biosystems, Foster City, Calif.) according to the manufacturer's instructions. Quantitative real-time polymerase chain reaction (qRT-PCR) was performed using the reverse transcription reaction product, TaqMan MicroRNA Assay kit, and TaqMan Universal PCR Master Mix (Applied Biosystems) according to the manufacturer's instructions. TaqMan MicroRNA Assay kits for human miRs were used. Reactions were loaded onto a 96-well plate and run in duplicate on an ABI 7500 Fast Real-Time PCR System (Applied Biosystems). The reactions were incubated at 50° C. for 20 seconds and 95° C. for 10 minutes, followed by 40 cycles of denaturation at 95° C. for 15 seconds, then 1 minute of annealing/extension at 60° C. The ΔΔC_{T} method was used to determine relative number of copies (RQ) of miR. Data were normalized to a C. elegans synthetic miR sequence, cel-miR-39 (Qiagen), which was spiked in as a control during RNA isolation. The miScript PCR System from Qiagen (Valencia, Calif.) was used for quantification of miR-142-5p and miR-92b. miRs were isolated as described previously; 5 μL of isolated template RNA were used for subsequent reverse transcription reactions which were performed using the miScript II RT Kit according to the manufacturer's instructions. Real-time PCR was performed using 2× QuantiTect SYBR Green PCR Master Mix, 10× miScript Universal Primer, 10× miScript Primer Assay, and template cDNA from reverse transcription; all reaction volumes suggested by the manufacturer were doubled to perform reactions in duplicate.

### Results

### Tumor miR Array

Thirty three enucleated uveal tumors with monosomy-3 and 22 without were profiled. SAM was used to identify differentially expressed miRs with the false discovery rate set to Q=0.001. This analysis identified 26 miRs as discriminators; 19 were down-regulated two-fold and six were up-regulated two-fold (Table 1). In this data set, 13 patients had manifested metastatic disease clinically on follow-up. Most of the miRs identified were also differentially expressed in these patients (Table 1). The strongest associations with monosomy-3 were observed for underexpression of X-linked miRs. None of these miRs, nor any other miRs, were differentially expressed by tumors from the 31 males compared to the 24 females studied. Tumor infiltrating lymphocytes (TILs) in uveal melanoma tumors are associated with a poor prognosis and are a potential source of miRs. Singh et al., Melanoma Res.; 11:255-63 (2001). Twenty seven of the tumors evaluated were considered to have TILs; 30 were not. None of the miRs differentially expressed in tumors with and without monosomy were differentially expressed in tumors with and without TILs.

TABLE 1
Tumor miRs differentially expressed

| | Chro- | | | Plasma Levels* | |
|---|---|---|---|---|---|
| | mo- | | Metas- | Monoso- | Disomy |
| miR | some | P | tasis | my 3 | 3 |

Over-expressed in tumors with monosomy-3

| | | | | | |
|---|---|---|---|---|---|
| hsa-miR-135a* | 3 | 0.003 | NS^{†} | ND^{‡} | ND |
| hsa-miR-624 | 14 | 0.003 | 0.02 | ND | ND |
| hsa-miR-449b | 5 | 0.005 | NS | ND | ND |
| hsa-miR-142-5p | 17 | 0.006 | NS | 1236 | 1059 |
| hsa-miR-92b | 1 | 0.009 | NS | 688 | ND |
| hsa-miR-628-5p | 15 | 0.01 | NS | ND | ND |

Under-expressed in tumors with monosomy-3

| | | | | | |
|---|---|---|---|---|---|
| hsa-miR-509-3-5p | X | 0.00000008 | 0.01 | ND | ND |
| hsa-miR-508-3p | X | 0.0000003 | 0.02 | ND | ND |
| hsa-miR-514 | X | 0.0000009 | 0.04 | ND | ND |
| hsa-miR-506 | X | 0.000001 | 0.04 | ND | ND |
| hsa-miR-513a-5p | X | 0.000002 | 0.01 | ND | ND |
| hsa-miR-507 | X | 0.000003 | 0.02 | ND | ND |
| hsa-miR-509-3p | X | 0.000004 | 0.04 | ND | ND |
| hsa-miR-513b | X | 0.0003 | 0.001 | ND | ND |
| hsa-miR-876-3p | 9 | 0.0003 | NS | ND | ND |
| hsa-miR-378* | 5 | 0.002 | NS | ND | ND |
| hsa-miR-935 | 19 | 0.003 | 0.05 | ND | ND |
| hsa-miR-181a | 9 | 0.004 | NS | 546 | ND |
| hsa-miR-99a | 21 | 0.009 | NS | ND | ND |
| hsa-miR-194 | 1 | 0.01 | NS | ND | ND |
| hsa-miR-592 | 7 | 0.01 | NS | ND | ND |
| hsa-miR-1296 | 10 | 0.01 | NS | ND | ND |
| hsa-miR-624* | 14 | 0.02 | NS | ND | ND |
| hsa-miR-140-5p | 16 | 0.02 | NS | ND | ND |
| hsa-miR-651 | X | 0.02 | NS | ND | ND |

*Average signal intensity
^{†}NS = not significant
^{‡}ND = not detectable

### Tumor miR Biogenesis

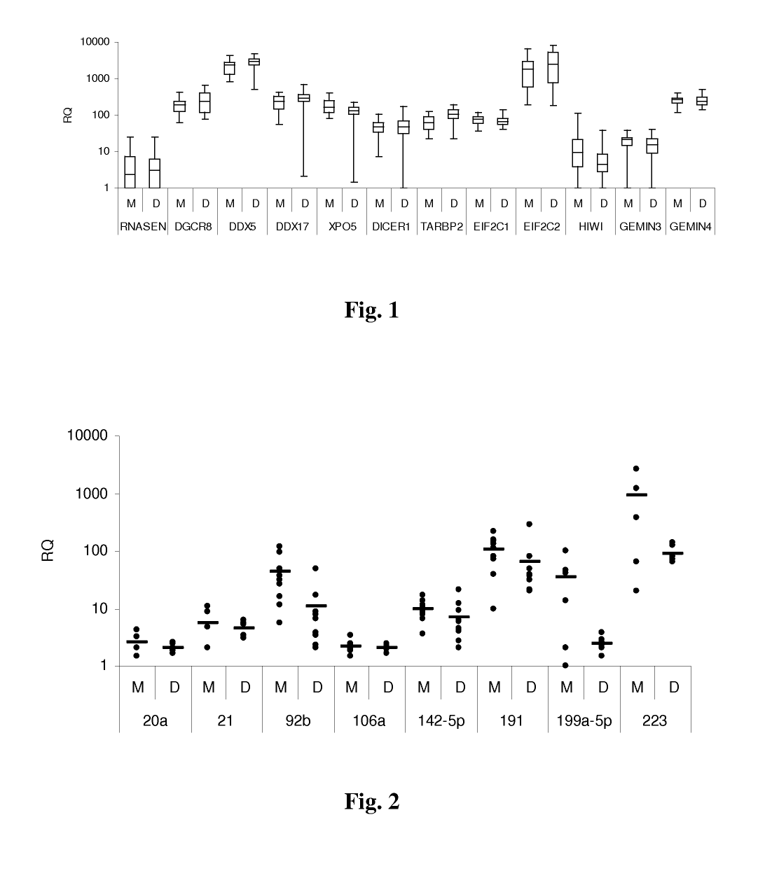

Twelve miR biogenesis factors were also profiled in the 55 enucleated uveal melanoma tumors. Tumors manifesting monosomy-3 were characterized by lower levels of TARB2 and DDX17 and higher levels of XPO5 and HIWI (FIG. 1).

### Plasma miR Array

Plasma miR profiles of pooled samples from 10 patients with monosomy-3 and 10 without were analyzed using qNPA array. Of the 674 human miRs assayed, 96 were detectable in plasma. Compared to patients without, 11 miRs were elevated two-fold and four were reduced two-fold in patients with tumor monosomy-3 (Table 2). None of the miRs that were discriminatory in the tumor array were discriminatory in the plasma array. Only two of the miRs overexpressed in the tumor array were measurable in blood, miR-92b and 142-5p (Table 1). The 1.6 fold increase in plasma miR-92b was statistically significant (p<0.02); the 1.2 fold increase in plasma miR-142-5p was not (p<0.5) was not. The only other miR measurable in blood was miR-181a, levels of which were increased in plasma while being underexpressed in tumors in the presence of tumor monosomy-3.

TABLE 2
Plasma miRs differentially expressed

| | | | | |
|---|---|---|---|---|
| miR | Chromosome | Monosomy 3 | Disomy 3 | P |

Increased in patients with tumor monosomy-3

| | | | | |
|---|---|---|---|---|
| hsa-miR-191 | 3 | 7456 | 1760 | 0.0000001 |
| hsa-miR-93 | 7 | 3344 | 836 | 0.000001 |
| hsa-miR-221 | X | 10411 | 3449 | 0.00006 |
| risa-miR-342-3p | 14 | 962 | ND^{†} | 0.00007 |
| hsa-miR-19b | 13 | 2385 | 1017 | 0.0002 |
| hsa-miR-199a-5p | 19 | 1977 | ND | 0.0003 |
| hsa-miR-25 | 7 | 1490 | ND | 0.0009 |
| hsa-miR-27a | 19 | 5182 | 1993 | 0.0009 |
| hsa-miR-23a | 19 | 4566 | 1886 | 0.001 |
| hsa-miR-15b | 3 | 1195 | 530 | 0.001 |
| hsa-miR-223 | X | 10286 | 3413 | 0.002 |

Decreased in patients with tumor monosomy-3

| | | | | |
|---|---|---|---|---|
| hsa-miR-1227 | 19 | 1686 | 10791 | 0.0000008 |
| hsa-miR-663 | 20 | 2196 | 16206 | 0.00001 |
| hsa-miR-654-5p | 14 | 420 | 1148 | 0.00008 |
| hsa-miR-1238 | 19 | 1561 | 6172 | 0.0001 |

*Average signal intensity
^{†}ND = not detectable

### Plasma miR Quantification

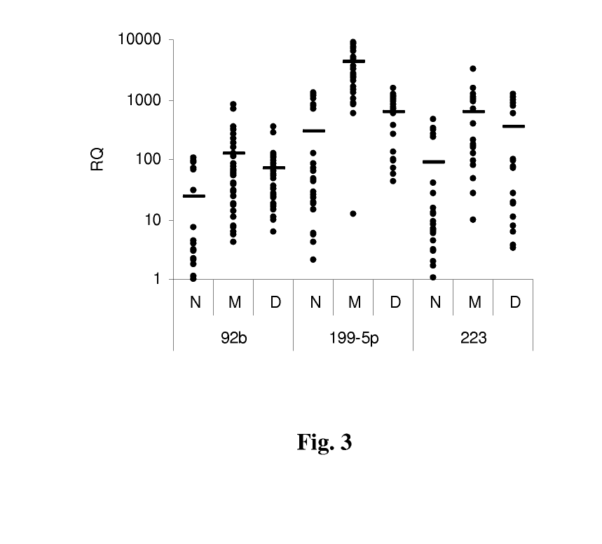

Plasma levels of select miRs increased in the tumor and in the pooled plasma arrays in the presence of tumor monosomy-3 were then examined by qRT-PCR in the individual patients tested, again 10 with tumor monosomy-3 and 10 without. The focus was on the two miRs that were over-expressed in the tumor array that were measurable in plasma, miR-92b and 142-5p, and three miRs elevated in the plasma array, miR-191, 199a-5p, and 223. Three miRs previously reported to be upregulated in uveal melanoma tumors compared to normal choroid, miR-20a, 21, and 106a, that were not differentially expressed in either the tumor or plasma array, were also assessed. Yang et al., Sci China Life Sci.; 54:351-8 (2011). Differential expression in plasma by qRT-PCR paralleled the qNPA array results (FIG. 2). miR-92b, 199a-5p and 223 were significant higher in both the qNPA array and the qRT-PCR analysis. miR-191 tended to be higher in the qRT-PCR analysis, but increases did not reach the level of significance (P<0.10), as it did in the qNPA array analysis. miR-142-5, 20a, 21, and 106a were not differentially expressed. Levels of the three miRs that were significantly different were then examined in another set of patients with primary uveal melanoma in which tumor chromosome 3 status was obtained on FNA biopsies. Levels of these miRs were also compared to those of 26 healthy donor controls. Plasma levels of miR-92b, 199a-5p, and 223 were significantly higher in patients with monosomy 3 when compare to patients with disomy; levels of all three were also higher when compared to levels of normal controls (FIG. 3).

### DISCUSSION

With the goal of developing prognostic biomarkers, miR expression levels were examined in patients with uveal melanoma. Of 858 miRs assessed, six were found to be overexpressed and 19 underexpressed in tumors manifesting monosomy-3, an accurate predictor of the development of metastasis. None of the miRs differentially expressed in the tumors studied have been previously associated with uveal melanoma. The miRs associated with monosomy-3 were analyzed by DIANA mirPath (Multiple microRNA Analysis), a web-based application. Papadopoulos et al., 25:1991-3 (2009). They were found to be potential regulators of actin cytoskeleton, adherens junctions, and TGF-beta signaling, pathways implicated in metastasis, including in uveal melanoma. Makitie et al., Invest Ophthalmol Vis Sci.; 42:2442-9 (2001); Chang et al., Melanoma Res.; 18:191-200 (2008); Woodward et al., Invest Ophthalmol Vis Sci.; 43:3144-52 (2002). None were also identified as being differentially expressed by tumors studied by Worley et al., who examined 470 miRs and used the class 2 gene expression profile as a surrogate for metastasis. Worley et al., Melanoma Res.; 18:184-90 (2008). miR-135a, associated with metastasis in hepatocellular carcinoma, and miR-449b, associated with invasion in endometrial cancer, were overexpressed in tumors with monosomy-3. The most significant discriminators were underexpression of miRs of the 506-514 cluster, which has been implicated in melanoma development and invasiveness. Streicher et al., Oncogene.; 31:1558-70 (2012). Zhao et al., Toxicol Lett.; 205:320-6 (2011). miR-509 was the most significantly downregulated miR in cutaneous melanoma nodal metastases. Caramuta et al., J Invest Dermatol.; 130:2062-70 (2010). miR-506 may function as a tumor suppressor. Also underexpressed were miR-142-5p, underexpression of which in gastric and germ cell cancers is associated with a worse prognosis; and miR-181a, which may function as a tumor suppressor in glioma.

Tumors manifesting monosomy-3 were characterized by alterations in miR processing factors, which have been associated with several types of cancer, suggesting that these can control metastasis-initiating events. TARB2 and DDX17 were upregulated, and XPO5 and HIWI were downregulated. TARBP2 (12q12-q13) has been implicated in the development of colorectal and other cancers. Melo et al., Nat Genet.; 41:365-70 (2009). DDX17 (22q13.1) was found to be a renal cell carcinoma metastasis-associated gene. Tan et al., Int J Cancer; 123:1080-8 (2008). It is also overexpressed in colon carcinoma. In breast cancers, increases in DDX17 were associated with a favorable prognosis. XPO5 (6p21.1) has been shown to be down-regulated in lung adenocarcinoma. Chiosea et al., Cancer Res.; 67:2345-50 (2007). In contrast, XPO5 is upregulated in prostate and breast adenocarcinomas. Chiosea et al., Am J Pathol.; 169:1812-20 (2006). Alterations in HIWI (12q24.33) have been linked to germ cell tumors. Qiao et al., Oncogene; 21:3988-99 (2002). Alterations in Dicer, Drosha, and Gemin4, which have been observed in cutaneous melanoma, were not observed. Most of the miRs that were discriminatory in tumors with monsomy 3 were downregulated.

Using the tumor and plasma arrays as guides, the inventors found specific miRs to be significantly increased in the plasma of patients with tumor monosomy-3. These included one overexpressed in the tumor array, miR-92b, and two increased in the plasma array, 199a-5p, and 223. Plasma levels of miRs reported to be upregulated in uveal melanoma tumors compared to normal choroid, miR-20a, 21, and 106a, were not discriminatory. Virtually all of the miRs discriminatory in the tumor array were not measurable in the plasma array. In contrast to tumor, more miRs were differentially increased/overexpressed in the plasma in patients with tumor monosomy-3 compared to without. That miR-expression patterns of tumor differ from those of blood has been previously reported. Pigati et al., PLoS ONE.; 5:e13515 (2010). Several mechanisms may generate blood miRs, including passive leakage from apoptotic or necrotic cells and active secretion of miR-containing microparticles and exosomes. These can occur in malignant cells but also in nonmalignant cells with a short half-life, such as blood cells, or upon tissue damage. There is evidence that most circulating miRs are blood-cell derived. Mitchell et al., Proc Natl Acad. Sci. USA.; 105:10513-18 (2008). At least 100 different miRs have been shown to circulate in the blood of healthy donors, including most of the miRs the inventors found to be differentially increased: miR-19b, 191, 199a-5p, 25, 23a, 223, and 93. Chen et al., Cell Res.; 18:997-1006 (2008). Zhao et al., PLoS ONE.; 5: e13735 (2010). Several of the differentially expressed miRs identified have been previously reported to be elevated in the plasma or serum of patients with cancer, including miR-92b in prostate (Lodes et al., PLoS One.; 4:e6229 (2009)); miR-223 in lung, esophageal, and hepatocellular; miRs-19b, 199a-5p, 15b, and 25 in lung. Chen et al., Int J Cancer.; 130:1620-8 (2012).

In addition to chromosome 3, abnormalities in chromosomes 1, 6, and 8 have also been associated with metastasis in uveal melanoma. Only three of the 26 tumor miRs and two of the 18 plasma miRs differentially expressed are located on these chromosomes. The miR-506-514 cluster maps to the human X chromosome, which contains approximately 10% of all miRs detected so far in the human genome. miR-223 and miR-221, which were increased in plasma, are also X-linked. Although the role of most has not yet been described, several X-linked miRs have been shown to have important functions in cancer as well as in immunity. Nonrandom abnormalities have been previously observed on the sex chromosomes in uveal melanoma, but a consensus regarding their prognostic significance has not been established. White et al., Cancer Genet Cytogenet.; 170:29-39 (2006). Although males manifest a slightly higher incidence and mortality rate, gender is not considered to play a major role in uveal melanoma predisposition or prognosis. Singh et al., Ophthalmology.; 118:1881-5 (2011).

These results support the analysis of miRs and also miR biogenesis factors as biomakers of uveal melanoma metastatic risk. They also suggest that potentially useful blood miRs may be derived from the host response as well as the tumor. Tumor monosomy 3, although an accurate predictor, is a surrogate endpoint of metastatic death. Prospective studies with clinical endpoints are in progress. Optimization of the testing platforms is also desirable. Although the results of the qNPA array paralleled the results of qRT-PCR analysis for most of the plasma miRs, some discrepancies were observed that may be due to differences in detection sensitivity and probe designs for array, as well as the different normalization processes: microarray uses a global normalization for comparison across the samples, whereas qRT-PCR data were normalized using a single reference. Several other approaches are under investigation to assess circulating miRs, including approaches that involve isolating circulating tumor cells. Mostert et al., Expert Rev Mol Diagn. 11:259-75 (2011). In addition to further studying prognostic significance, studies wherein blood miR levels are being assessed in conjunction with imaging studies as part of systemic surveillance for metastases in patients with defined tumor genotypes are underway. Finally, miRs are under investigation as therapeutic targets. Jackson A, Linsley P S., Discov Med. 9:311-8 (2010). Further testing in uveal melanoma of miRs is also warranted in this regard.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of providing a prognosis for a subject having uveal melanoma, comprising:
obtaining a biological sample from the subject;
determining the expression level of one or more uveal melanoma-associated miRs and/or miR biogenesis factors in the biological sample; and
characterizing the subject as at an increased risk if one or more uveal melanoma-associated miRs and/or miR biogenesis factors are differentially expressed as compared with a corresponding control.

2. The method of claim 1, wherein the biological sample is a tumor sample.

3. The method of claim 2, wherein the miR is selected from the group consisting of miR-135a, miR-624, miR-449b, miR-142-5p, miR-92b, and miR-628-5p, and the miR is over-expressed as compared with the corresponding control.

4. The method of claim 2, wherein the miR is selected from the group consisting of miR-509-3-5p, miR-508-3p, miR 514, miR-506, miR-513a-5p, miR-507, miR-509-3p, miR-513b, miR-876-3p, miR-378, miR-935, miR-181a, miR-99a, miR-194, miR-592, miR-1296, miR-624, miR-140-5p, and miR-651, and the miR is under-expressed as compared with the corresponding control.

5. The method of claim 1, wherein the biological sample is blood, blood plasma, or blood serum.

6. The method of claim 5, wherein the miR is selected from the group consisting of miR-191, miR-93, miR-221, miR-342-3p, miR-19b, miR-199a-5p, miR-25, miR-27a, miR-23a, miR-15b, and miR-223, and the miR is over-expressed as compared with the corresponding control.

7. The method of claim 5, wherein the miR is selected from the group consisting of miR-1227, miR-663, miR-654-5p, and miR-1238, and the miR is under-expressed as compared with the corresponding control.

8. The method of claim 1, wherein the expression level of a plurality of uveal melanoma-associated miRs is determined.

9. The method of claim 8, wherein the expression level of the miRs is determined using a quantitative nuclease protection assay.

10. The method of claim 1, wherein expression of the one or more miRs is determined using the quantitative real-time polymerase chain reaction.

11. The method of claim 1, wherein a miR biogenesis factor is detected, the biogenesis factor is TAR RNA binding protein 2 or p72 and the factor is upregulated, or the biogenesis factor is Exportin 5 or HIWI protein and the factor is downregulated, as compared with a corresponding control.

12. The method of claim 1, further comprising the step of providing suitable treatment if the subject is identified as being at increased risk.

13. The method of claim 12, wherein the treatment is radiation therapy.

14. A method of monitoring the development of metastases in a subject having uveal melanoma, comprising:
obtaining a blood, blood plasma, or blood serum sample from the subject;
determining the expression level of one or more uveal melanoma-associated miRs in the sample; and
characterizing the subject as having an increased risk of developing metastases if one or more uveal melanoma-associated miRs are differentially expressed as compared with a corresponding control.

15. The method of claim 14, wherein the miR are selected from the group consisting of miR-20a, miR-125b, miR-146a, miR-155, miR-223, miR-199a, and miR-181a.

16. The method of claim 14, wherein the subject has received anticancer treatment prior to the step of obtaining a sample.

17. A kit for providing a prognosis for a subject having uveal melanoma, comprising
one or more probes capable of determining the expression level of one or more uveal melanoma-associated miRs and/or miR biogenesis factors in a biological sample, and a package for holding the one or more probes.

18. The kit of claim 17, wherein the kit further comprises instructions for using the kit to carry out a method of providing a prognosis for a subject having uveal melanoma.

19. The kit of claim 17, wherein the kit comprises a plurality of probes and a microarray.

20. The kit of claim 17, wherein the kit comprises one or more probes capable of determining the expression level of a miR selected from the group consisting of miR-135a, miR-624, miR-449b, miR-142-5p, miR-92b, and miR-628-5p, miR-509-3-5p, miR-508-3p, miR 514, miR-506, miR-513a-5p, miR-507, miR-509-3p, miR-513b, miR-876-3p, miR-378, miR-935, miR-181a, miR-99a, miR-194, miR-592, miR-1296, miR-624, miR-140-5p, and miR-651.

21. The kit of claim 17, wherein the kit comprises one or more probes capable of determining the expression level of a miR selected from the group consisting of miR-191, miR-93, miR-221, miR-342-3p, miR-19b, miR-199a-5p, miR-25, miR-27a, miR-23a, miR-15b, and miR-223, miR-1227, miR-663, miR-654-5p, and miR-1238.

22. The kit of claim 17, wherein the kit comprises one or more probes capable of determining the expression level of a polynucleotide expressing a miR biogenic factor selected from the group consisting of is TARB2, DDX17, XPO5 and HIWI.
